SEQUENCE LISTING (7 sequences)

SEQ ID NO: 1 - DNA, 25 bp, Human immunodeficiency virus
aatggcagtattcatccacaatttt

SEQ ID NO: 2 - DNA, 26 bp, Human immunodeficiency virus
gtctactattctttcccctgcactgt

SEQ ID NO: 3 - DNA, 16 bp, Human immunodeficiency virus
aaagaaaaggggggat

SEQ ID NO: 4 - DNA, 63 bp, Artificial
  sequence of detection of ACV
agtattcatccacaattttaaaagaaaaggggggattggggggtacagtgcaggggaaagaat

SEQ ID NO: 5 - DNA, 63 bp, Artificial
  Sequence of calibration of ACV
agtattcatccacaattttaaaagggggaaaaggattggggggtacagtgcaggggaaagaat

SEQ ID NO: 6 - DNA, 15 bp, Artificial
  Region of the probe in the wild virus
aagaaaaggggggat

SEQ ID NO: 7 - DNA, 16 bp, Artificial
  Region of the probe of the mutated virus
aaagggggaaaaggat

(19) United States
(12) Patent Application Publication
(10) Pub. No.: US 2010/0291538 A1
(43) Pub. Date: Nov. 18, 2010

(54) ARTIFICIAL CALIBRATION VIRUS TO CONTROL HIV VIRAL LOAD TESTS BY PCR IN REAL TIME

(75) Inventors: Antônio Gomes Pinto Ferreira, Rio de Janeiro (BR); José Antönio Pinto de Sá Ferreira, Rio de Janeiro (BR); Amilcar Tanuri, Rio de Janeiro (BR); Rodrigo de Moraes Brindeiro, Rio de Janeiro (BR); Patricia Alvarez Baptista Brindeiro, Rio de Janeiro (BR)

Correspondence Address: MEREK, BLACKMON & VOORHEES, LLC, 673 S. WASHINGTON ST., ALEXANDRIA, VA, 22314, US

(73) Assignee: FUNDACAO OSWALDO CRUZ-FIOCRUZ, Rio de Janeiro (BR)

(21) Appl. No.: 12/225,179

(22) PCT Filed: Mar. 16, 2007
(86) PCT No.: PCT/BR2007/000066

§ 371 Date: Jul. 30, 2010

(30) Foreign Application Priority Data
Mar. 16, 2006 (BR) PI 0600715-5

(51) Int. Cl. C12Q 1/70; C12N 7/01

(52) U.S. Cl. 435/5; 435/235.1

(57) ABSTRACT

The present invention refers to the design of an artificial calibrating virus (ACV), as well as a methodology quality guarantee system, which has controlling characteristics in the performance of all the stages carried out during a detection and/or quantification molecular test. More specifically, the referred to ACV is used for the validation and calibration of quantitative determinations of circulating viruses in blood plasma samples by means of polymerase chain reaction (PCR) technology in real time (or ‘real time PCR’).

### INVENTION FIELD

The present invention refers to the construction of an artificial calibrating virus (ACV), as a system of methodological quality guarantee, which has controlling characteristics in the performance of all the stages executed during the detection molecular test and/or viral quantification. More specifically, the referred to ACV is used for the validation and calibration of quantitative determinations of circulating viruses in blood plasma samples by means of the polymerase chain reaction technology (PCR) in real time (or “real time PCR”).

The deposits of plasmid pZ2Z6 and virus VCA-Z2Z6 were made at the American Type Culture Collection—ATCC, and PTA-6609 (plasmid) and PTA-6610 (virus) were identified.

### STATE OF THE ART

Historically, in the state of the technique, the concentration of HIV viral particles in the blood plasma of infected patients is determined by means of the culture viral technique, in which the dosage of the levels of viral protein p24, antigen, is performed, or evaluation of the impact on the counting of TCD4⁺ lymphocytes in the cells.

At an initial phase of the infection caused by the HIV virus, the levels of the TCD4⁺ lymphocytes in the cells are near the values of normality; however, the detection of viral protein p24 is not possible yet, that is, the antigen. It is believed that the HIV virus at this stage is in a reduced stage of multiplication.

The detection of high viral levels of HIV is related with the drop in the number of TCD4⁺ lymphocytes in the cells and with the emergence of symptoms associated, to the diseases caused by the Acquired Immune-Suppressant Deficiency Syndrome, AIDS. In the period when there is a drop in the number of TCD4⁺ lymphocytes, antigen p24 is easily detected and because of this, the HIV virus can be isolated in culture.

With the advent of the polymerase chain reaction (PCR), it was possible for the first time to quantify the number of copies of nucleic acids, RNA or DNA, in most of the infected individuals. The method developed in the polymerase chain reaction (PCR) has a high level of sensitivity and permits detecting up to 10² virions/ml in blood plasma.

Thus, the development of several commercial assays began, which were capable of establishing the intensity of the viral multiplication during the clinical latency period of the HIV virus and the monitoring of the response to the anti-retroviral therapy.

At present, in the state of the technique 3 commercial products are known, which use distinguished methods to quantify the viral load of the HIV. The best results for these assays are obtained for viruses of the HIV-1 type. This type of virus is found prevailingly in countries of the First World, as well as also in Brazil, country in which the present invention was developed. The commercial products known today are:

RT-PCR Technology→Amplicor HIV-1 Monitor (Roche);

NASBA Technology→Nuclisens HIV-1 QT (Organon Teknika/BioMerieux); and

BDNA→Quantiplex HIV-1 RNA 2.0 Assay (Chiron Corporation)

Statistical comparisons performed between the referred to 3 commercial products, disclosed a close correlation (above 90%) between the 3 assays; however, the product called Amplicor presented, in general, higher values in the analyses of the tests performed. The analysis of the intra-test variation did not disclose systematic differences between the duplicates with any of the 3 products mentioned, which suggests the good reproducibility of the same. Product Quantiplex HIV RNA 2.0 Assay presented lesser discrepancies without, however, compromising the reproducibility of the kit.

Quantiplex when compared with the two other methods uses a volume of sample that is higher and demands more time to perform, since it includes 3 stages more than the other two methods, which need approximately 8 hours for the full performance of their 4 main stages.

The most adequate material to be used in the 3 different methodologies already known is comprised of blood plasma samples, which contain the target virus, in this case, the HIV. The blood plasma must be obtained and right after that, it must be purified so as to separate the HIV virus from the other RNAs or other cellular components.

The purification of the target virus should occur as soon as possible after its achievement. After its purification, the virus can be stored for 1 or 2 days at an approximate temperature of 25° C. or even for weeks, at an approximate temperature of 4° C. The use of anti-coagulant or inhibiting substances can lead to the emergence of secondary products during the performance of the method; therefore, one must abide by instructions of the manufacturer of the product to be used.

For illustration purposes, a brief summary of the methodologies used in the 3 commercial products known in the state of the technique is described.

### Amplicor HIV-1 Monitor (Roche)

The methodology used by product Amplicor HIV-1 Monitor, includes the direct amplification of a specific region of the strip of complementary DNA nucleic acid generated by the reverse transcription, which covers a target sequence of 142 pairs of bases situated on the region of the gag gene of the HIV-1 by means of the polymerase chain reaction (PCR).

The patent request PI9800337-2, describes the methodology followed by the technology of product Amplicor HIV-1 Monitor. Said methodology enables the generation of multiple copies of a specific sequence of nucleotides of a given organism and includes a cycle made up by 3 stages, which repeat themselves several times during the entire reaction. The reaction is performed in a thermocycler, an equipment that controls and varies automatically the temperatures in scheduled periods of time (cycles), by a defined number of 30 to 40 cycles. The stages comprising one cycle are:

denaturation;

hybridization, and

extension

The stage of denaturation occurs by means of the heating of the sample at temperatures above 90° C. The complementary DNA of double strip is separated into two simple strips.

The hybridization stage consists of the interaction of the primers (initiators) with each one of the complementary simple DNA strips obtained at the denaturation stage, in a range of temperature between 40° C. and 65° C. At this stage, there occurs a target sequence of approximately 100-600 pairs of bases, which is specific for each type of microorganism.

The extension stage occurs under a preferred temperature of 72° C. This stage needs the help of a specific thermo-resistant enzyme, polymerase DNA. This enzyme has the capability of synthesizing new DNA molecules of double chains identical to the target region, of 142 pairs of bases, as of the region delimited by the primers, initiators. Two new strips of DNA similar to the original target sequence are generated and the finalization of the cycle then happens, which is started again and repeated several times.

### Nuclisens HIV-1 QT (NASBA—Akzo Nobel/Organon Teknika)

This methodology describes the direct amplification process of the gag gene region of the HIV nucleic acid. The amplification process is isothermal and continuous. The referred to process makes use of synthetic RNAs as internal reaction controllers and the detection of the HIV virus RNA happens by means of the electrochemoluminiscence technique.

In this methodology, the internal calibrators include a group of 3 synthetic RNAs (Qa—high concentration, Qb—medium concentration, Qc—low concentration) that are distinguished from the HIV RNA (wild) in a sequence of 20 nucleotides, located in the central part of the region of the gag gene to be amplified.

The methodology developed by product Nuclisens HIV-1 QT involves the following stages:

release;

isolation of the viral RNA;

amplification, and

detection of the material amplified.

At the stage of release and isolation of the viral RNA, the plasma or serum samples are lisated in an appropriate solution, which contains a mixture of guanidine tiocyanate and triton×100 solution. This solution provides the solubilization of proteins and lipids, deactivation of infectious agents and present enzymes, disintegration of the viral particles and release of nucleic acid.

The stage of viral isolation occurs by means of the addition of the internal calibrators and the silica under conditions of high saline concentration. Said particles will be binding to the nucleic acids (RNA) of the calibrators and the plasma or serum sample and after several washes of the nucleic acid, said acid is eluted.

The stage of amplification occurs in the presence of an initiator (P1), which contains the site of recognition of an enzyme, the T7-RNA polymerase, which favors its binding to the target sequence of the HIV-1 RNA. With the help of a reverse transcriptase, there occurs the extension of P1 contributing for the formation of a DNAc. The activity of a third enzyme, RNAseH, eliminates the RNA molecule of the hybrid DNA-RNA and the presence of a second initiator (P2) permits the synthesis of the second filament of DNA with the help of the reverse transcriptase and, from then on, has as sequence the synthesis of new molecules of DNA, in a cyclic way.

For the detection of amplified material, aliquots of the amplified samples, are added to a hybridization solution, which contains a specific generic probe, marked with ruthenium, for each one of the RNAs, bound to the magnetic spheres by complex streptoavidine-biotin. With the help of a magnet on the surface of an electrode, there is attraction and immobilization of the magnetic particles, which are washed by means of a buffer solution, which permits the elimination of the free RNA.

When one applies a tension over the electrode, a reaction of electrochemoluminescence is triggered, which provides the quantification of the amplified sample and transmitted to a photo-multiplier tube. After converting it into digital signal, its interpretation is done by a software of reader Nuclisens that draws a standard curve as of the reading of the 3 calibrators (Qa, Qb and Qc) and, thus, it is inferred in the curve the reading obtained for the wild RNA, which permits the establishment of the concentration in copies/mL of the viral RNA.

### Quantiplex HIV RNA 2.0 Assay

This method begins with the precipitation of the HIV virus as of the centrifuge technique of the blood plasma sample of an individual infected by the HIV virus. After the centrifugation of the sample of blood plasma, there occur the lisate of the cells and the release of the viral genomic RNA.

The viral genomic RNA is transferred to the microcavities of a board where it will be captured by a set of target probes of specific synthetic oligonucleotides.

The viral genomic RNA and the pre-amplifying probes, which are complementary to another fraction of the genome of the HIV virus, are hybridized with a second set of single amplifying probes (branched DNA). Each set of target probes is connected to different regions of the target gene of the viral RNA.

In order to amplify the signal, multiple copies of a probe marked with alkaline phosphatase are hybridized to the immobilized probe. The incubation of this complex with a chemoluminescent substrate provides detection conditions, since the light emitted is directly proportional to the quantity of HIV present in the sample and registered through luminescent counts by a board reader.

The concentrations of the HIV-1 are determined in accordance with a standard curve defined by the emission of light as of standard solutions containing concentrations known of a recombinant bacteriophages.

However, the commercial cost of the use of the methodologies described in the state of the technique is, to say the least, the double of the commercial cost of the use of the technique that counts T CD4+ lymphocytes in the cells.

Table 1 shows, as an illustration, a comparative picture of the 3 commercial products known in the state of the technique.

TABLE 1
Main points on the different methodologies.

| Needs/characteristics of the | PCR | Nuclisens | Quantiplex |
|---|---|---|---|
| methodologies | Amplicor | NASBA | bDNA |
| Use of thermocycler | X | | |
| Thermocycler for | | | |
| quantification in real time | | | |
| Automatic analyzer | X | | |
| (photometer) | | | |
| Reader of chemoluminescence | | X | X |
| High speed refrigerated | X | | X |
| centrifuge | | | |
| Synthetic RNAs such as | X | X | |
| internal calibrators | | | |
| Use of VLP as internal | | | |
| calibrator | | | |
| Polymerase DNA enzyme for | X | | |
| transcription and extension | | | |
| RNAse, T7-RNA enzymes, reverse | | X | |
| transcriptase | | | |
| Conjugate and substract for | X | | |
| disclosure of the reaction | | | |
| Initiators for the detection | | X | |
| stage | | | |
| “Probes” of oligonucleotides | | | X |
| Pre-amplifying “Probes” | | | X |

The intrinsic characteristics of the immunological response of each individual to the report, ever more frequent, of the development of resistance to the anti-retroviral drugs, have contributed to reinforce the need of a more reliable test and one with more impact on the monitoring of the infection by the HIV if compared to the count of T CD4+ lymphocytes in the cells.

Along with the determination of T CD4+ lymphocytes in the cells, the quantification of the viral load of the HIV has been, for some years now, one of the instruments that we have been used to monitor infection caused by HIV. The quantification of the viral load provides information on the risk of disease progression, on the appropriate occasion to start therapy, on the level reached of anti-retroviral activity and on the effectiveness of any given therapeutic regimen.

In addition to these technologies that have been used recently, other methodologies have been recently developed. These new methodologies are based on the technique of “Real Time PCR” and “molecular beacons” for the quantification of the viral load of the HIV of a patient infected with this virus. However, these new methodologies have not yet incorporated the idea of using an “artificial calibrating virus” (ACV) to control the stage of HIV purification in the plasma of the patient and extraction of its RAN.

Up to the present time, no kit developed and/or patented for the quantification of the viral load of the HIV has used a virus of the ACV to control all the stages in the viral quantification process, from the stage of viral concentration and extraction up to the detection of the circulating viral load in the serum-positive HIV-1 individual. The kits existing in the market do not monitor the viral extraction stage, since they use, as control, a synthetic RNA or plasmid, generally added during the amplification phase of the viral RNA. The ACV proposed in the present invention is biosafe and permits controlling all the stages of the technology, because it is added at a concentration defined to the plasma of the patient before the quantification of the viral load of the HIV-1 gets started. In addition, the ACV can be used to correct the viral load of the patient obtained in the same sample.

### SUMMARY OF THE INVENTION

The present invention is based on the amplification of a viral gene by polymerase chain reaction (PCR) in real time, also called “real time—PCR”. The viral genome (RNA) of the virus isolated from the patient's plasma is initially submitted to a synthesis reaction of strips of complementary DNA (DNAc) to the genome, by means of the reverse transcription technique.

### BRIEF DESCRIPTION OF THE DRAWINGS

FIG. 1A shows the stages of the reaction of PCR in real time for the quantification of viral load of HIV-1 using an internal calibrating virus.

FIG. 1B is a chart of the concentration curve of product synthesized as a result of the number of cycles performed during the PCR reaction.

FIG. 2 is a schematic representation of the HIV-1 genome (infective clone Z6Δnefgpt), where FIG. 2A represents the proviral clone of the HIV-1 originated from construct Z6 Δ nef gpt. FIG. 2B represents the env gene deactivated by the insertion of the nucleotide T and FIG. 2C represents the site-directed mutations in the gene of the integrase, generating the ACV.

FIG. 3 is a chart of the amplification curve of pNL43 different concentrations.

FIG. 4 is a chart of the amplification curve of the calibrator for the previous standard curve.

FIG. 5 is a chart of the standard external calibration curve (standard curve), drawn by a computer software.

FIG. 6 displays dispersion and Pearson correlation charts for each pair of methodologies coupled (validation with clinical samples).

FIG. 7 displays dispersion and Pearson correlation charts (correlation between coupled methods).

### DETAILED DESCRIPTION OF THE INVENTION

For the amplification of a viral gene by polymerase chain reaction (PCR) in real time, in accordance with the invention, the viral genome (RNA) of the virus isolated from the plasma of a patient is initially submitted to a reaction of strip synthesis of complementary DNA (DNAc) to the genome, by means of the reverse transcription technique. Part of the viral integrase gene is amplified by PCR in real time with the use of specific oligonucleotide initiators (flanking “initiators”). A PCR in real time is a direct quantification technique of the amplified DNA, which uses the reading of fluorescence as of fluorophores incorporated to a complementary nucleotide probe (or that hybridizes) to the center of the amplified DNA region of the reaction (or amplicon of the reaction). The period of time required—measured in cycles of the amplification—for the reaction to reach approximately 50% of its efficiency is the parameter used to obtain the concentration relative of viral genome present at the beginning of the methodology. For such, the presence of an internal reaction calibrator is necessary, which provides a standard of amplification in real time corresponding to the number previously known of RNA molecules of this calibrator.

In addition, said calibrating virus, in addition to quantifying the test sample, the calibrator also has the role of validating each determination, since a deviation from the value observed with regard to that expected—which is outside a statistical range of safety—can be used to invalidate this determination.

In general, this internal calibrator (or calibrators) can be considered because of these two reasons as fundamental to quantitative determination. The correct drawing of this calibrator and its bio-construction are fundamental for the success in the development of the methodology of determination of viral load.

A few considerations must be taken into account for this development:
a) The calibrator must present a structure homologous that that which we intend to detect (the genome of the virus in the patient's plasma) and be included in the reaction in a previously established concentration, which must be confirmed by the same methodology, which the present virus in the patient's plasma is submitted to.
b) Because it is a validator of the determinations, this calibrator must be introduced in the human plasma sample to be tested, at the beginning of the test, and be present in all of its stages, so as to be a control of the variations in the different methodologies. The same, therefore, must behave as the target material of the determination, which is the virus and its genome in RNA, suffering all the methodological interventions of the latter, up to the final stage of amplification in real time and quantification. Therefore, for a better identification with the target of the determination (the patient's virus) the calibrator must also be a virus with RNA genome; preferably an HIV-1.
c) The genome of this virus HIV-1 must be presented modified (mutated in the laboratory). Said virus generated by artificial mutation is used as calibrator of a reaction in parallel with the natural virus, so that it can be innocuous in its manipulation, without replicate capability in vivo and, therefore, not infectious. Said characteristics take place by means of a deletion in part of its genome, so as not to change its structure and its physical-chemical characteristics.
d) Since the calibrator is also an HIV virus, the same gets confused, when mixed with the test sample, to the very virus present in the sample (to be quantified). To avoid this to happen, the calibrator must be mutated artificially in its sequence of the integrase gene, which is the target of the hybridization by the disclosing fluorescent probe of the reaction. By mutating only this target, keeping the binding capability of the amplifying oligonucleotides (initiators), both genes of the integrase of the virus of the sample and calibrator can be amplified by PCR using the same system (control of parameters of the reaction). The distinction between the two viruses is done by the presence of two different probes coupled to different fluorophores. However, the amplification and the detection will be differentiated because there is no complementarities (capacity to hybridize) of bases between the natural sequence and the artificial sequence, which was generated with the use of the two oligonucleotides, which were different and specific for each one of the sequences so as to permit that they are used in the same detection system, Taqman, Hairoin oligoprobes, Scorpion primers, Sunrise primes or any other. Therefore, all the methodological and physical-chemical conditions of the reactions are kept, both for the wild virus and for the artificial calibrator.
e) Since this viral artificial calibrator, with identification with the HIV-1 will be going through all the chemical and biochemical processes that the sampling virus will also go through, the amount of this calibrator, which previously was known as the one to be used in the reaction, should not interfere with the efficiency of the amplification itself/detection of the sampling virus, in view of the identification of the two of them. More serious should be the evaluation of the interference of the fixed quantity of the calibrator with the sample virus, when this has a very low viral load (next to the limit of detection) or very high (which would cause the sample to interfere, as a result of the excess, with the determination of the calibrator, leading to the invalidation of the determination).

The present invention consists of the development of an Artificial Calibrating Virus (ACV), which is a reaction controller during the performance of the stages of extraction, purification and viral amplification during the quantification test of viral load of the HIV of a sample of blood plasma of a patient infected by said virus. More specifically, the ACV is a virus that has a target sequence of the probe in little variable regions, from the genetic point of view. Said regions encompass the gag region, the RT region, the integrase region, among others.

For the development of the ACV, it was necessary to create a semi-automated viral load test and a wide range of detection. Thus, it was chosen to use the PCR in real time technique, which enables the dosage of the PCR products during the course of the reaction, since it is a direct quantification technique of the amplified complementary DNA molecule.

The dosage of the PCR products occurs by means of a probe of Synthetic DNA, which has modifications at its ends. At its 5′ ends, a molecule is incorporated that emits fluorescence, which is absorbed by another molecule located at end 3′.

During the polymerization promoted by enzyme rTth DNA, the fluorescent molecule located at the end 5′ is removed due to the activity of a specific enzyme, 5′ exonuclease, so as to emit light. This emission of light is directly proportional to the quantity of product mass of PCR that is being synthesized. Due to this factor, it is possible to dose in real time the PCR product.

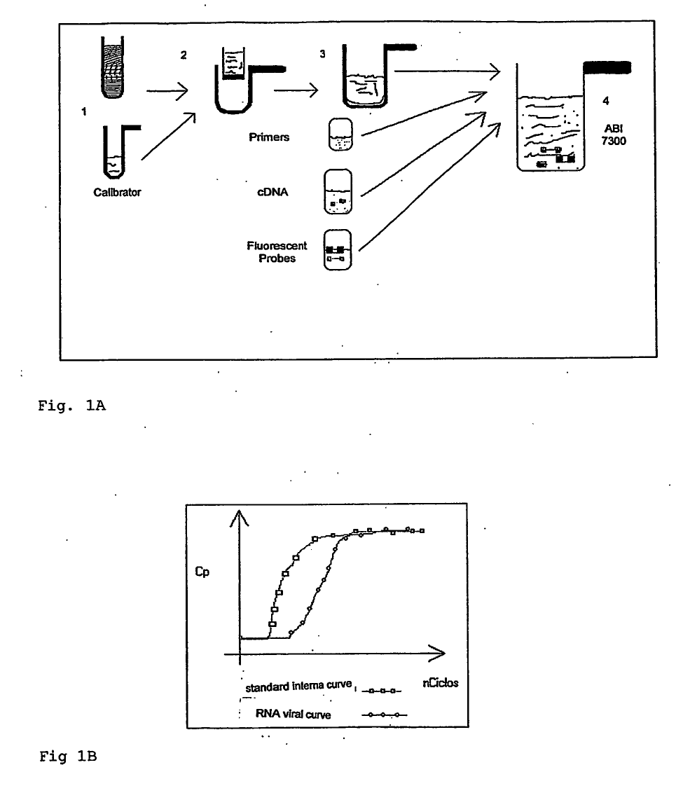

This technology speeds up the initial RNA quantification by means of a curve of accumulation of PCR product. This way, the larger the quantity of initial RNA in the RT-PCR reaction, the lower will be the number of cycles for the beginning of the amplification exponential phase (Ct). FIG. 1 illustrates the stages developed for the direct quantification of the PCR products. Said stages include:

1) A sample of plasma from a patient infected by the HIV-1 virus is isolated and a calibrating virus representing a structure homologous to that which one intends to detect (the genome of the virus in the sample of patient's plasma) is introduced in the reaction at a concentration previously established, which must be confirmed by the same methodology, to which are submitted the viruses present in the patient's plasma sample. In the present achievement, one used a calibrating virus at the preferred concentration of 10^{4.}

2) The sample mixture of plasma+VCA has its RNA extracted by means of a Kit of extraction Qiagen® or by means of any other commercial methodology of RNA extraction.

3) The viral RNA+VCA are used in the PCR in real time reaction, which uses a thermocycler ABI 7000.

4) During the PCR in real time reaction, a curve of concentration of synthesized is generated as a result of the number of cycles performed during the PCR reaction. It is based on this curve that the number of the reaction molecules is estimated.

In the assembly of this new methodology, several technological developments were required, among them, the definition of the genomic region of the HIV-1 for the drawing of the PCR initiators.

The target genomic region is a genetically stable region and its complementary initiators are capable of amplifying any isolate of the HIV-1 virus from the M group. In the present achievement, the target genomic region chosen was the C-terminal portion of the integrase gene. This portion is genetically stable and its initiators had already been previously tested in preliminary studies.

In Table 2 a sequence of specific oligonucleotide initiators is found (flanking primers) of the integrase gene, which can be drawn and tested at the early stage of the methodology described in the present invention.

The initiators were estimated as of the alignment of sequences from the M group. The consensus of the sequences shows that this genomic region is very preserved and the three primers (downstream SEQ ID NO. 1), reverse (SEQ ID NO. 2), and the fluorescent probe SEQ ID NO. 3)) were drawn to recognize the target region and present a temperature of denaturation or “melting” (Tm) in the range of 58-65° C.

TABLE 2
Sequences of the integrase enzyme gene regions,
which will serve as the base for the drawings of
the probes.

| | | Probe FAM | |
|---|---|---|---|
| | Primer advance | (SEQ ID NO. | Reverse Primer |
| | (SEQ ID NO. 1) | 3) | (SEQ ID NO. 2) |
| Sequence | AATGGCAGTATTCA | AAAGAAAAGGG | GTCTACTATTCTTT |
| 5′-3′ | TCCACAATTTT | GGGAT | CCCCTGCACTGT |

Another methodology developed for the present invention was the adequate drawing and the construction of the artificial calibrating virus (ACV).

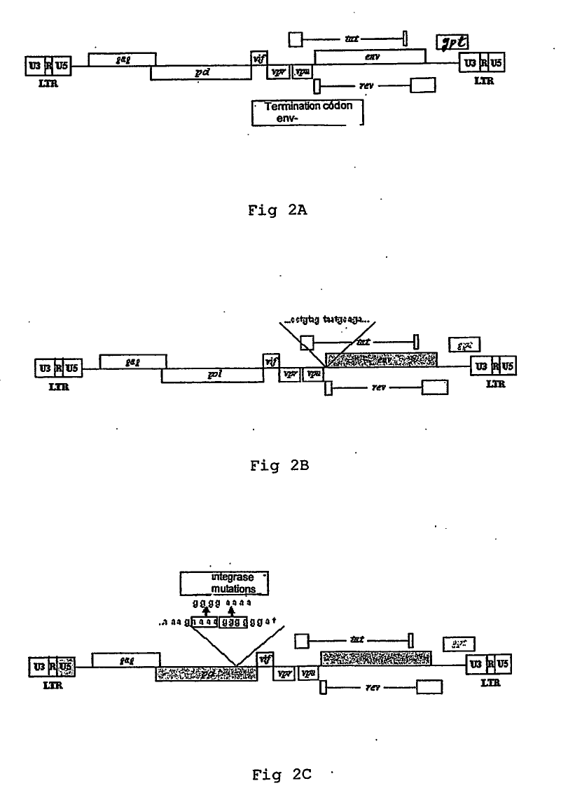

The ACV serves to control the inter-assay variation, to validate the runs, in addition to correcting the viral load obtained with the patient's plasma sample virus. This virus should be the exact copy of the same genomic region of the target PCR. This is due to the use of an infective clone of the HIV-1 virus, such as for instance, Z6Δnefgpt, which has the gene of xanthene-guanine phosphoribosyl transferase (gpt) of E. coli, in the place of Nef, as shown in FIG. 2A; and an insertion of a nucleotide (T) which changes the reading phase of gene env, so as not to permit the expression of the proteins of the envelope, as shown in FIG. 2B. On the construction of a calibrating virus, reference is made to Tanuri A et al's article, Construction of a selectable nef-defective live-attenuated human immunodeficiency virus expressing Escherichia coli gpt gene, Virology. 2000 Mar. 1; 268(1): 79-86. So, changes were also made in the target sequence in the integrase gene of this clone, using the site-directed mutagenesis technique by complementary initiators, as shown in FIG. 2C.

This ACV virus was selected because it was not infectious, that is, bio-safe and provide the dosage of the viral load, by means of comparative analyses, since there is no change in its morphogenesis. For the production of the VCA in the present achievement, we used cells COS7; however, we could have used any other type of permissive cellular lineage. Cells COS7 were transfected with about 2 μg of the infective clone modified to generate the calibrating viruses by means of sprouting (release of the virus through the cellular membrane) some 72 hours after its transfection.

When 2 μg of the infective clone is transfected in cells COS7 by means of the complexation with cationic liposome's, such as, for example, the lipofectina® (Invitrogen, USA) are generated around 10⁷ viral particles/mL of culture supernatant. The ACV simply forms particles without envelope proteins (gp120/gp41), since the gene of the envelope was inactivated, thus not being infectious. This fact confers a level of extra biological safety to the process, which is basic when manipulating the ACV in clinical laboratories.

Table 3 presents the modification introduced into the ACV genome in the region of the integrase gene, in which region 3′ terminal is represented. The first 19 bases and the last 19 bases both of the sequence (SEQ ID NO. 4) of detection and in the sequence of calibration (SEQ ID NO. 5), indicate the sequences of PCR in real time reaction initiator oligonucleotides.

Table 4 presents the region of detection with the different probes for virus HIV/wild (SEQ ID NO. 6) and for the calibrating virus. The mutated sequence (SEQ ID NO. 7) in the ACV prevents the crossed complementarities of the probes with the different viral gene targets, so as not to change the identical complexity of both sequences, in order to generate the same Tm for both sequences.

The differentiation between the wild virus and the ACV is determined due to the mutagenesis occurred. In Table 3, such a mutagenesis is identified in the range as of the 25th base up to the 32nd base in the calibration sequence. In Table 4, such a mutagenesis is identified in the range as of the 5th base up to the 12th base in the region of the probe in the mutated virus.

TABLE 3
Target sequences of the integrase gene of HIV-1

DETECTION SEQ
(SEQ ID NO. 4)
5′AGTATTCATCCACAATTTTAAAAGAAAAGGGGGGATTGGGGGGTACAG
TGCAGGGGAAAGAAT3′
CALIBRATION SEQ
(SEQ ID NO. 5)
5′AGTATTCATCCACAATTTTAAAAGGGGGAAAAGGATTGGGGGGTACAG
TGCAGGGGAAAGAAT3′

TABLE 4
Sequences of the molecular probes.

| | Region of the | Region of the |
|---|---|---|
| | probe in the | probe in the |
| | wild virus | mutated virus |
| | (SEQ ID NO. 6) | (SEQ ID NO. 7) |
| Sequence 5′-3′ | AAGAAAAGGGGGGAT | AAAGGGGGAAAAGGAT |

The invention will now be described in greater detail by means of the following examples, which should not be interpreted as restrictive to the scope of the invention.

### Example 1

### Preparation of a Standard External Curve

A viral isolate of subtype B, pNL43 present in a sample of blood plasma, was previously prepared in culture of cells susceptible to the elaboration of a curve of dilution of the virus at previously known concentrations. In the present achievement, a stock of this HIV-1 of subtype B was prepared in a viral concentration known of approximately 8×10⁹ copies of viral RNA/mL of culture supernatant (confirmed by commercial tests NASBA Nuclisens, Cosba Amplicor and b-DNA).

A standard curve was prepared, which was prepared by means of the dilution of a standard virus (“standards”) at the approximate concentrations of: 10⁶, 10⁴ and 10² copies of viral RNA/mL of supernatant. This standard curve should follow the viral load determination assay, so as to serve as an external calibrating curve, or a standard curve (“Standard curve”) of the PCR in real time reaction.

### Example 2

### Purification and Extraction of the Viral RNA

The isolation of the nucleic acid RNA is performed by means of a commercial kit in high conditions of denaturation for the deactivation of a few enzymes, such as for example, the RNAses, so as to warrant the integrity of the RNA isolated. In the present achievement, it was used the kit QIAamp Mini Viral RNA, owned by Qiagen. The concentration of the saline solution and the pH of the buffer solution used during the cellular lise permit the connection of the RNA in the membrane of silica-gel of the extraction column. In the present achievement, it was used a QIAamp® column. Only the RNA connects with the membrane, being removed the contaminating substances by means of two washings with other types of buffer solutions. These buffer solutions used to wash do not interfere in the binding of the RNA in the membrane. The purified RNA is free from proteins, nucleases and other contaminants and inhibitors. The extraction, by means of the extracting column QIAamp® is an easy and efficient methodology because it warrants the integrity and purity of the RNA.

The protocol of the reaction for the extraction and purification of the viral RNA includes the following stages:

Separate the cellular components of the blood plasma sample of the infected patient by the HIV by means of the centrifugation technique, in adequate equipment at approximately 3500 rpm for an approximate period of 10 minutes;

Remove an aliquot of about 1 mL of the patient's plasma and transfer it to a collecting recipient, preferably an Eppendorf tube;

Add about 100 μL of the calibrating virus ACV to the Eppendorf tube, which contains the, patient's plasma (corresponding to 12750 copies of the virus ACV/mL final) and 1 mL of each sample of the standard curve of dilution of the HIV at the approximate concentrations of: 10⁶, 10⁴ and 10² copies of RNA viral/mL;

Separate the mixture by the centrifugation technique for approximately 2 hours at a preferred temperature of 4° C. at the approximate speed of 14000 rpm. The Eppendorf tubes must have been previously marked so as to facilitate the visualization of the pellet;

Carefully remove approximately 960 μl of the supernatant so that the pellet is not perforated;

Add approximately 560 μl of the buffer solution AVL, carrier of the nucleic acid so that cellular lise occurs to the pellet and stir the solution in an adequate equipment; for example, a vortex for approximately 15 seconds;

Incubate the mixture for approximately 10 minutes in the flow where the extraction is being performed, at an approximate temperature of 25° C.;

Add approximately 560 μl of alcoholic solution; for example, ethanol (100% or 96%);

Stir the solution briefly with a final volume of approximately 1260 μl in an adequate equipment, such as, for example, a vortex and then centrifuge the solution for: approximately 15 seconds at a preferred speed of 8000 rpm;

After the centrifugation, add to the extraction column, approximately half of the volume (630 μl) of said solution, couple the column to the collecting tube that is enclosed with the commercial extraction kit (QIAamp® Viral RNA Mini Kit);

Centrifuge said column at a preferred temperature of 25° C. for an approximate period of 1 minute at an approximate speed of 6000×g (8000 rpm), discard the collecting tube and couple a new collecting tube;

Add the remaining 630 μl of the solution and centrifuge again at a preferred temperature of 25° C. for an approximate period of 1 minute at 8000 rpm, discard the collecting tube and couple a new collecting tube to the column;

add to the column approximately 500 μl of the buffer solution of washing 1, which is attached to the commercial extraction kit, centrifuge the coupled column to the collecting tube at a preferred temperature of 25° C. for an approximate period of 1 minute at an approximate speed of 6000×g (8000 rpm), discard the collecting tube, which contains the eluted material and couple a new collecting tube to the column;

add to the column approximately 500 μl of the buffer solution of washing 2, which goes with the commercial extraction kit, centrifuge the column coupled to the collecting tube at a preferred temperature of 25° C. for an approximate period of 3 minutes at an approximate speed of 20000×g (14000 rpm), discard the collecting tube, which contains the eluted material and couple a new collecting tube to the column;

centrifuge the column coupled to the collecting tube at a preferred temperature of 25° C. for an approximate period of 1 minute at an approximate speed of 6000×g (8000 rpm), discard the contents of the collecting tube recoupling the same to the collecting tube;

couple the extraction column to a tube preferably of 1.5 ml, add approximately 50 μl of water/DEPC, centrifuge at a preferred temperature of 25° C. for approximately 1 minute at an approximate speed of 6000×g (8000 rpm);

remove the column and store it at approximately −70° C. in the 1.5 ml tube the RNA eluted from the column.

Before beginning the extraction one must follow a few procedures so that the extraction is as efficient as possible: (i) one must initially perform a rinsing of the tube, (ii) let the carrier buffer solution at room temperature so that the temperature is stabilized and thus warrant the absence of crystals in solution, since the crystals can inhibit the extraction of the nucleic acid, RNA. The buffer solution bearer of the present invention is the buffer solution present in the commercial kit QIAamp Mini Viral RNA, owned by Qiagen. If there is the presence of crystals in the buffer solution, it is necessary to heat the buffer solution at a range of approximate temperature of 60 to 65° C. for an approximate period of 3-5 minutes, until the buffer solution is translucid.

### Example 3

### Reverse Transcription of the Extracted RNA

This methodology warrants the efficiency of the complementary DNA synthesis (DNAc) by means of the use of random initiators (or random primers). The achievement of the DNAc is performed preferably in final volume of 50 μL, of which around 25 μL (half of the reaction volume) must be of RNA. The reverse transcription is performed in the presence of a specific enzyme, such as, for example, the MuLV/RNAseH enzyme at the approximate minimum concentration of 50 U/uL, in a buffer solution 10× of the RT, 25× dNTP and 10× “Random Primers”.

In Table 5 below, the substances used during the composition of the mixture of reagents used during the reverse transcription are listed.

TABLE 5
Substances used in the composition of the mixture
of reagents used in reverse transcription.

| | 1 sample | | 31 samples | |
|---|---|---|---|---|
| 10X RT Buffer | 5 | uL | 155 | uL |
| 25X dNTP mix | 2 | uL | 62 | uL |
| 10X Random Primer | 5 | uL | 155 | uL |
| Multi Scribe RT | 2.5 | uL | 77.5 | uL |
| (50 U/ul) | | | | |
| H₂O RNAse-free | 10.5 | uL | 325.5 | uL |
| Final volume | 25 | uL | 775 | uL |

The procedure for the technical performance of reverse transcription must be performed so as to abide by the following protocol:

add approximately 25 μl of RNA to a tube preferably of 0.2-1.5 ml.

incubate the reaction at a preferred temperature of 25° C., in a heating block or in a PCR device for an approximate time period of 10 minutes.

after the performance of the previous stage, incubate the reaction at a preferred temperature of 37° C. in a heating block or in a PCR device for an approximate time period of 2 hours.

store the complementary DNA achieved in a collecting tube, such as for example, an Eppendorf tube, at a preferred cooling temperature of −20° C.

### Example 4

### Reaction of Quantitative PCR

The advantage in using the two-stage RT-PCR is that each reaction is performed separately. The main benefits of this methodology are: (1) Different primers can be used for the RT stage and for the PCR stage, which helps preventing errors in the binding of primers and permits the maximum use of the hot start of AmpliTaq Gold® DNA Polimerase. (2) The aliquot of remaining DNAc of an RT-PCR can be used for a new analysis, if necessary.

For the preparation of the reaction board, duplicates must be made of the standard curve and the negative control (all the reagents, without the addition of DNAc). The final volume of each reaction is of approximately 25 μL. In the present achievement, the sample board was fully filled out, that is, the 8 rows and the 12 wells. The procedure for this methodology followed the protocol described below:

1. thaw and homogenize all the reagents described in Table 5;

2. prepare the mixture with the reagents described in Table 5, in a clean area of amplicon DNA;

3. schedule the reaction sequence (application of the samples on the board) in the thermocycler;

4. prepare the PCR stock mixture for N (=number of reactions)×25 μl (volume of the reaction);

| | 1 | | 100 | |
|---|---|---|---|---|
| | Reaction | | Reactions | |
| 20X working stock Assay Mix | 1.25 | uL | 125 | uL |
| 2X Taq Man Universal Master | 12.5 | uL | 1250 | uL |
| Mix | | | | |
| Calibrator-VIC | 1.8 | uL | 180 | uL |
| H2O | 4.45 | uL | 445 | uL |
| Volume of the Mix | 20 | uL | 2000 | uL |
| DNAc | (5 | uL) | | |
| Final volume of each | (25 | uL) | | |
| Reaction | | | | |

5. the collecting tube containing the reagent mixture must be stirred constantly so that the bubbles are eliminated. The stirring should occur in appropriate equipment, such as for instance, a mini-centrifuge;

6. add approximately 20 μl of the stock mixture to the bottom of each one of the 96 wells of the sample board;

7. add approximately 5 μl of water in the negative control (NTC) or sample to the appropriate reaction tube. Prepare the negative control first, and then the samples of patients.

8. after the addition of the PCR reagent mixture, one must add 5 μl of complementary DNA relative to each sample to the board well of the samples, with ascent and descent movements to homogenize with the pointer the samples with the reaction mixture, completing the final volume of 25 μl;

9. seal the reaction board with an adhesive seal or optical covers. The reactions are now ready for cycling.

10. briefly centrifuge the sealed board in adequate equipment, such as for example, a centrifuge, for approximately 30 seconds at a preferred speed of 20000×g (14000 rpm), so as to eliminate all the bubbles;

11. place the board in the thermocycler (ABI Prism 7000) and close the cover of the equipment. The reading of the reaction board is done in a platform ABI 7000 of Real Time PCR, for fluorescence FAM and VIC, in accordance with the manufacturer's instructions.

### Example 5

### Calculation of the Amount of Copies in the Reaction

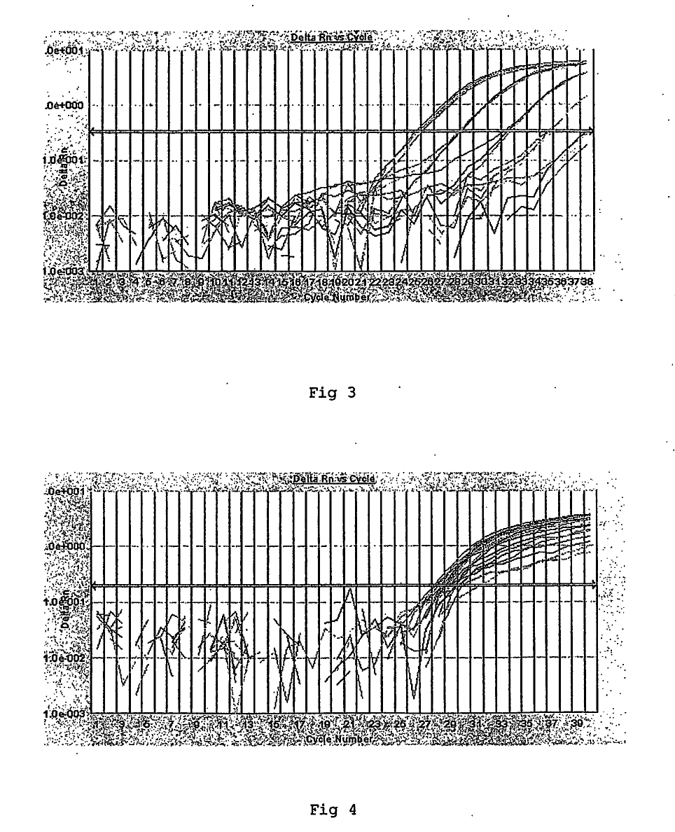

The computer program (software) provided by ABI (Applied Biosystems Inc, USA) on its platform of Real Time PCR 7000, provides the user with a file of results in the format of a Microsoft Excel® worksheet. A sequence of Macros (software protocols) was developed during the present invention in order to process the data obtained during the reaction de RT-PCR. As of the standard curve, of known viral RNA concentrations (10⁶, 10⁴ and 10² copies of the viral RNA/mL), a semi-logarhythmic regression was made of its concentrations (axis y) against the Ct (period of reaction, measured in PCR cycles, in which the amplification of the material sample starts). This curve is possible to be assembled thanks to the previous knowledge of the ACV values of the standard curve. As of this, it will be possible to obtain the ACV values of all sample tests, in accordance with their time of amplification (Ct) measured by the nadir of the exponential increase of the fluorescence emitted by the FAM fluorophore, in accordance with FIG. 3.

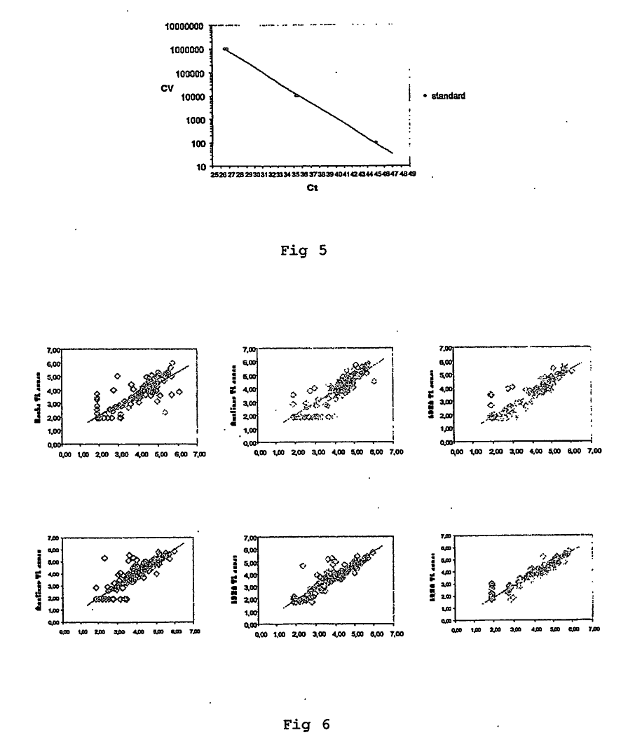

It is at this stage that it is of the utmost importance the internal reaction calibrator: since this “contaminates” each determination due to the 12750 copies/mL of this ACV to each sample and its number of cycles (Ct) is detected by an emission of different fluorophore (VIC) the curves for this specific fluorescence, in all of the sample determinations and the standard curve must have the same average of values in number of cycles (Ct) with a small standard deviation (generally varying in two cycles of PCR before and after the average number of cycles), in a normal distribution, as shown in FIG. 4.

What the software does is to calculate the average of the number of cycles of the artificial calibrator virus of each well. This average of the number of cycles must correspond to 12750 copies/mL in the regression of the external standard curve. If this does not happen, the value of the linear coefficient is modified to this condition of relation of the internal calibrator, considering that the new curve moves in parallel with the initial regression (the angular coefficient is not modified since the error must be in the course of all the concentration ranges of the viral load, given the linearity of the correlation between the viral load concentration and the number of cycles). FIG. 5 shows the standard external calibration curve (standard curve) drawn by the software developed.

As of the new formula of the curve corrected of semilogarythmic regression of viral load concentration X number of cycles, the numbers of the cycles of each sample, at the VIC fluorescence of the internal calibrator, are analyzed for their location in the area of the regular curve of distribution of the Cts of VIC. Any sample that has a standard deviation from the average of the Cts of VIC>than 1.96 SD (95% CI) is considered invalid and its determination must be repeated. All the other samples that are found within an acceptable range of the standard deviation in relation to the average of Ct of the internal calibrator, will now have their viral load value calculated as of the corrected formula of regression of the standard curve (“standard”) (corrected by the average of the Ct do calibrator ACV).

### Example 6

### Preliminary Data of the Use of the ACV in the Determination of Viral Load (VL)

### Sensitivity

Fifty samples were repeated in groups of 10 repetitions, for the dilutions of a standard virus (NL4-3) of 200, 100, 80, 60, 40 and 20 copies of RNA/mL; for the calculation of the “cut off”, per distribution of Poison. The kit presented sensitivity in the range of 80 copies of RNA/mL of plasma, with >95% of specificity in the range. A Table 6 shows the sensitivity data of the detection limit of 20/40/60/80/100/200 copies of HIV-1/mL.

TABLE 6
Sensitivity data of detection limit of
20/40/60/80/100/200 copies of HIV-1/mL.

| | | |
|---|---|---|
| Samples | | |
| copies/mL | Viral load | log |

| | | | |
|---|---|---|---|
| 20 | A | NR | NR |
| | A | 2.17 | 0.34 |
| | B | 0.08 | −1.12 |
| | B | NR | NR |
| | C | <detection | <detection |
| | | limit | limit |
| | C | 1.35 | 0.13 |
| | D | 4.93 | 0.69 |
| | D | <detection | <detection |
| | | limit | limit |
| 40 | A | <detection | <detection |
| | | limit | limit |
| | A | <detection | <detection |
| | | limit | limit |
| | B | <detection | <detection |
| | | limit | limit |
| | B | 38.10 | 1.58 |
| | C | 3.98 | 0.60 |
| | C | <detection | <detection |
| | | limit | limit |
| | D | <detection | <detection |
| | | limit | limit |
| | D | 0.52 | −0.28 |
| 60 | A | 19.18 | 1.28 |
| | A | 79.29 | 1.90 |
| | B | 47.47 | 1.68 |
| | B | 3.67 | 0.57 |
| | C | 5.30 | 0.72 |
| | C | 1.58 | 0.20 |
| | D | 71.27 | 1.85 |
| | D | 4.93 | 0.69 |
| 80 | A | 27.49 | 1.44 |
| | A | NR | NR |
| | B | <detection | <detection |
| | | limit | limit |
| | B | 3.05 | 0.48 |
| | C | 1.49 | 0.17 |
| | C | 3.55 | 0.55 |
| | D | <detection | <detection |
| | | limit | limit |
| | D | 40.72 | 1.61 |
| 100 | A | 3.90 | 0.59 |
| | A | <detection | <detection |
| | | limit | limit |
| | B | 160.68 | 2.21 |
| | B | <detection | <detection |
| | | limit | limit |
| | C | 25.04 | 1.40 |
| | C | 23.74 | 1.38 |
| | D | 14.31 | 1.16 |
| | D | 7.70 | 0.89 |
| 200 | A | 156.46 | 2.19 |
| | A | 187.30 | 2.27 |
| | B | 25.37 | 1.40 |
| | B | 26.94 | 1.43 |
| | C | 59.94 | 1.78 |
| | C | 112.13 | 2.05 |
| | D | 1.72 | 0.24 |
| | D | 323.46 | 2.51 |

Validation with Clinical Samples and Viral Panels.

190 samples of patients' blood plasma HIV+ were collected. The collections were performed in fourfold, during the viral load testing of the patients.

Four viral load tests were performed of the viral loads in the samples of the HIV+ patients' blood plasma. Said analyses performed included the methodologies of the state of the art, Nasba Nuclisens QT with extractor, Cosba Amplicor Monitor Standard 1.5, Quantiplex v3.0 and the method described in the present invention, RT-PCR. The results of these tests were analyzed jointly so as to obtain the correlation between the different types of tests, and the validation of the test of the present request.

The comparative analyses performed in the results obtained of the tests made by the methodologies of the state of the art and the method developed in the present invention presented very close correlation coefficients of Pearson after the linear regression of the result of each pair of methodologies compared. The results can be seen in FIG. 6.

### Correlation Between Paired Methods

In a second phase of the development of this methodology a multicenter study was performed with three laboratories, which use in their routines, the kits from Roche (Amplicor HIV-1 monitor®), Biomérieux (Nuclisens HIV-1 QT®) and those from Bayer (Versant HIV 3.0 bDNA®). In this second phase it was established that each participating laboratory should test its routine samples by the commercial kit and by the method described in the present invention, so as to enable a comparative study between the methodologies. Table 7 shows the values of the viral load quantification of the HIV-1 for two methodologies used (Roche and Bayer) and for the methodology developed in the present invention, PCR in real time, in samples from different subtypes of HIV-1.

The methodology of RT-PCR (Amplicor HIV-1 monitor) presents a minimum limit of detection of 400 copies/mL. The number of samples tested was 315, being 84 (26.6%) invalidated by the value of “Y”. From the 231 (73.3%) samples, 18 (5.7%) were invalidated by the calibrator (Bio-Manguinhos program) and 106 were below the sensitivity limit, remaining 107 samples (33.9%) for the performance of the comparative analysis (n=107).

For the methodology of Nasba (Nuclisens HIV-1 QT), which presents sensitivity of 100 copies/mL, 351 samples were used, of which 135 (38.5%), performed on 4 different boards, were invalidated by the rejection of “Y”. From the 216 (61.5%) analyzed by the method in the present invention (BioManguinhos/UFRJ), 38 (10.8%) were invalidated by the calibrator (Bio-Manguinhos program). With the exclusion of the samples below the sensitivity limit, the final sampling of this study was of 65 (18.5%) samples (n=65).

In the laboratory, where the method of the present invention was performed comparatively with the bDNA technique (Versant HIV 3.0 bDNA), which presents a sensitivity of 50 copies/mL, 507 samples were tested of which 146 (28.8%) were invalidated, due to the following reasons: 89 (17.5%) samples, on 3 different boards where the value of “Y” was rejected; 1 board with 54 (10.6%) samples, by the positivism of the negative control (NTC) and 37 (7.3%) invalidated by the calibrator (Bio-Manguinhos program). The final sampling of this laboratory was of 182 (n=182), without the samples that remained below the detection limit.

Table 7 shows the values obtained in the quantification of the viral load of the HIV-1 by two of the methodologies used (Roche and Bayer) and by the methodology developed in the present invention (PCR in real time), in samples of different HIV-1 subtypes.

VIral Load

| | | Bayer | | Roche | | RT-PCR | |
|---|---|---|---|---|---|---|---|
| Sample | Subtype Pro/RT | Copies/mL | Log | Copies/ mL | Log | Copies/mL | Log |
| 261/01 | F1/B | 8.910 | 3.95 | 4.578 | 3.66 | 3175.66 | 3.5 |
| 359/01 | F/B | 64800 | 4.81 | 22338 | 4.35 | 10532.81 | 4.02 |
| 993/01 | F/B | 10800 | 4.03 | 3606 | 3.56 | 5735.24 | 3.76 |
| 1285/00 | F/B | <400 | 1.9 | 323 | 2.51 | 32.47 | 1.51 |
| 1477/00 | F/B | 50100 | 4.7 | 10512 | 4.02 | 13261.96 | 4.12 |
| 2120/01 | F/F | 8870 | 3.95 | 4569 | 3.66 | 3679.4 | 3.57 |
| 2438/01 | F/B | 684 | 2.84 | 475 | 2.68 | 338.36 | 2.53 |
| 2454/01 | F/B | 2000 | 3.3 | 604 | 2.78 | 467.83 | 2.67 |
| 114 | C/C | 83800 | 3.66 | — | — | 3507.97 | 3.55 |
| 1484 | B/B | 52600 | 4.72 | 18141 | 4.26 | 16846.67 | 4.23 |
| 1513 | B/B | 129000 | 5.11 | 71573 | 4.85 | 41510.78 | 4.62 |
| 361 | B/B | 102000 | 5.01 | 74639 | 4.87 | 76836.27 | 4.89 |
| 575 | B/B | 1690 | 3.23 | 2245 | 3.35 | 1154.07 | 3.06 |
| 577 | B/B | 1960 | 3.29 | 2498 | 3.4 | 960.07 | 2.98 |
| 904 | B/B | 1680 | 3.23 | 1132 | 3.05 | 667.29 | 2.82 |
| 983 | B/B | 2100 | 3.32 | 3655 | 3.56 | 4454.67 | 3.65 |
| 994 | B/B | 11700 | 4.07 | 5069 | 3.7 | 5735.24 | 3.76 |
| 2459 | B/B | 74300 | 4.87 | 8917 | 3.95 | 10897.81 | 4.04 |

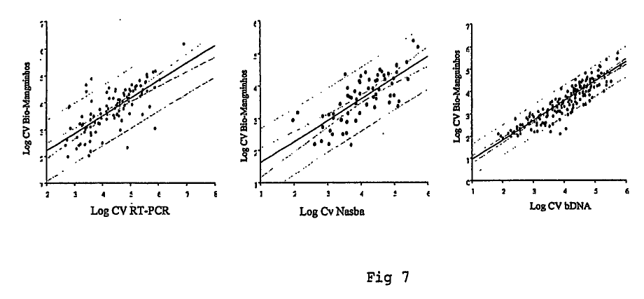

The correlation between the kits was evaluated according to the creation of a Cartesian chart of points (dispersion diagram), as shown in FIG. 7, and subsequent calculation of the simple linear regression and product-moment correlation coefficients (Pearson r) and of determination (r2). The values of the viral load found below the detection limit were not taken into consideration because they did not represent a real numeric value. The analysis of the correlations between the method described in the present invention and the commercial ones has shown that the same are significantly correlated (P<0,0001), as shown in Table 8. This correlation is considered very strong in relation to the bDNA and strong when compared with the NASBA and Amplicor.

TABLE 8
Relation in Log10 of the value of viral load in
copies/mL between the techniques P < 0.0001

| | No. de | Correlation | Value of t | Equation of the |
|---|---|---|---|---|
| Compared Kits | samples (n) | (r) | (tcalc) | straight line |
| RT-PCR × bDNA | 182 | 0.91 | 30.3 | y = 0.878x + 0.066 |
| RT-PCR × Nasba | 65 | 0.75 | 9.0 | y = 0.658x + 0.968 |
| RT-PCR × Amplicor | 107 | 0.71 | 10.59 | y = 0.651x + 0.909 |

### Reproducibility

For the determination of the inter-assay, inter-assay and inter-laboratory reproducibility, a panel with 3 different clinical samples was sent to the same 3 laboratories that participated in the correlation test, each one with the viral load previously determined (by the bDNA method) of 155, 323 and 475 copies of RNA/mL. Eight repetitions of each sample were tested on two different days (total of replicates=16) by two different operators (inter-assay).

Each one of the replicates was submitted in each laboratory to all the process of viral load analysis, since the viral isolation of the sample and the extraction of the RNA up to the detection in real time of the amplification of the target gene. This assay was called extraction reproducibility analysis, as shown in Table 9.

TABLE 9
Standard deviation of the extraction replicate determinations.

| | | intra- | Intra- | average | inter- | inter- |
|---|---|---|---|---|---|---|
| | | assay 1 | assay 2 | intra-assay | assay | laboratory |
| sample 1 | lab1 | 0.34 | 0.26 | 0.30 | 0.33 | 0.37 |
| (CV = 155) | lab2 | 0.18 | 0.36 | 0.27 | 0.28 | 0.37 |
| | lab3 | 0.31 | ND | 0.31 | ND | 0.37 |
| sample 2 | lab1 | 0.20 | 0.30 | 0.25 | 0.25 | 0.24 |
| (CV = 323) | lab2 | 0.37 | 0.16 | 0.26 | 0.28 | 0.24 |
| | lab3 | 0.13 | ND | 0.13 | ND | 0.24 |
| sample 3 | lab1 | 0.35 | 0.08 | 0.21 | 0.23 | 0.22 |
| (CV = 475) | lab2 | 0.32 | 0.13 | 0.22 | 0.27 | 0.22 |
| | lab3 | 0.11 | ND | 0.11 | ND | 0.22 |

CV = viral load in copies/mL
lab1 = Laboratory of Molecular Biology of the Hospital dos Servidores do Estado (HSE); this laboratory only analyzed seven replicates
lab2 = Laboratory of AIDS and Molecular Immunology - Fiocruz (LABAIDS)
lab3 = Laboratory of Viral Load, Hemocentro de Botucatu
ND = not determined, because this laboratory performed the reproducibility test on only one day

The 8 replicates of each one of the 3 samples were analyzed as to the extraction reproducibility, so that each laboratory (intra-assay and inter-assay) the standard deviation (evaluated as dispersion measure of the determinations) never exceeded 0.5 log (result of clinical significance). In each assay (intra-assay 1 and 2) different operators were responsible for the determinations.

In parallel to the reproducibility tests, one of the 8 replicates extracted by each laboratory on each execution day was submitted to 8 amplification reactions by PCR in real time (post-synthesis of DNAc), distinguished, for analysis of the variation (reproducibility) of this last methodological stage, being, therefore, called analysis of reproducibility of amplification, the data obtained in this analysis are shown in table 10.

TABLE 10
Standard deviation of the determinations of the
amplification replicates.

| | | intra- | intra- | average | inter- | inter- |
|---|---|---|---|---|---|---|
| | | assay 1 | assay 2 | intra-assay | assay | laboratory |
| sample 1 | lab1 | 0.37 | 0.74 | 0.56 | 0.59 | 0.43 |
| (CV = 155) | lab2 | 0.29 | 0.28 | 0.28 | 0.27 | 0.43 |
| | lab3 | 0.20 | ND | 0.20 | ND | 0.43 |
| sample 2 | lab1 | 0.14 | 0.23 | 0.18 | 0.20 | 0.23 |
| (CV = 323) | lab2 | 0.16 | 0.11 | 0.13 | 0.13 | 0.23 |
| | lab3 | 0.18 | ND | 0.18 | ND | 0.23 |
| sample 3 | lab1 | 0.29 | 0.10 | 0.20 | 0.38 | 0.40 |
| (CV = 475) | lab2 | 0.11 | 0.09 | 0.10 | 0.13 | 0.40 |
| | lab3 | 0.03 | ND | 0.03 | ND | 0.40 |

CV = viral load in copies/mL
lab1 = Laboratory of Molecular Biology of the Hospital dos Servidores do Estado (HSE); this laboratory only analyzed seven replicates.
lab2 = Laboratory of AIDS and Molecular Immunology - Fiocruz (LABAIDS)
lab3 = Laboratory of Viral Load, Hemocentro de Botucatu.
ND = Not determined, because this laboratory performed the reproducibility test in only one day.

During the repetition in 8 different reactions of amplification by PCR (amplification reproducibility) of a replicate, it was seen a deviation in one of the 3 laboratories of 0.74 in one of the determination assays (intra-assay 2). This punctual result, therefore, interferes in the result obtained inter-assay for this laboratory (0.59), being in the two cases in addition below log in difference. In the other 2 laboratories, this deviation did not happen, so as to be seen, therefore, as an intrinsic variation of this first laboratory (lab1), which does not reflect a characteristic of the methodology.

In the sample that presented the lowest viral load, it was seen, as expected, more intra-assay and inter-laboratory variations. However, this value remained always below the value of 0.5 log, of clinical significance.

In accordance with the comparative analyses performed between the commercial products in the state of the art and the methodology for the detection of viral load of the circulating HIV in patients described in the present invention, it is concluded that the present invention has detection limit sensitivity and specificity according to the values established for the commercial kits known in the stage of the art.

In preliminary results, the components of calibration/validation of the kit (internal calibrator—the internal calibrating virus, or ICV, and the external standard curve) presented good stability in a month at a −20° C. and −80° C., so that it needs continuity of this validation in periods of time higher than three, six and twelve months. All the other thermosensitive parts of the kit has already proven stable for more than six months at temperatures of −20° C.

As of the description presented herein, the improvement of the kit of the present invention was shown with regard to those known of the state of the technique (see Table 1), which may be summarized in the following way:

TABLE 11
Comparative chart of the 3 commercial products
known in the state of the technique and the present
invention.

| Needs/ | | | | PCR in real |
|---|---|---|---|---|
| characteristics of | PCR | Nuclisens | Quantiplex | time Bio- |
| the methodologies | Amplicor | NASBA | bDNA | Manguinhos |
| Use of thermocycler | X | | | |
| Thermocycler for | | | | X |
| quantification in real | | | | |
| time | | | | |
| Automatic analyzer | X | | | |
| (photometer) | | | | |
| Reader of | | X | X | |
| chemoluminescence | | | | |
| High speed | X | | X | X |
| refrigerated | | | | |
| centrifuge | | | | |
| Synthetic RNAs as | X | X | | |
| internal calibrators | | | | |
| Use of VLP as | | | | X |
| internal calibrator | | | | |
| DNA enzyme, | X | | | |
| polymerase for | | | | |
| transcription and | | | | |
| extension | | | | |
| RNAse enzymes, | | X | | X |
| T7-RNA, reverse | | | | |
| transcriptase | | | | |
| Conjugated and | X | | | |
| substrate for the | | | | |
| disclosing of the | | | | |
| reaction | | | | |
| Initiators for the | | X | | X |
| detection stage | | | | |
| “Probes” of | | | X | X |
| oligonucleotides | | | | |
| Pre-amplifying | | | X | |
| “Probes” | | | | |

The invention herein described, as well as the aspects covered here must be considered as one of the possible achievements. However, it must be clear that the invention is not limited to these achievements and those with skills in the technique will note that any particular characteristic introduced in it must be understood only as something that was described in order to facilitate understanding and cannot be done without moving away from the described inventive concept. The limiting characteristics of the object of the present invention are related with the claims that are part of the present report.

1. Artificial Calibrating Virus (ACV) generated by artificial mutation in the target sequence of the probe in little variable regions from the genetic point of view characterized as having SEQ ID NO. 7 sequence.

2. Artificial Calibrating Virus (ACV) in accordance with claim 1, characterized for being employed in the control of a quantification reaction of HIV viral load.

3. Artificial Calibrating Virus (ACV) in accordance with claim 1, characterized for being employed in the correction of the viral load of the viruses of infected individuals.

4. Artificial Calibrating Virus (ACV) in accordance with claim 1, characterized by the fact that the genomic region of the HIV used for its development being conservative and the primer downstream including SEQ ID NO. 1.

5. Artificial Calibrating Virus (ACV) in accordance with claim 1, characterized by the fact that the genomic region of the HIV used for its development being conservative and the reverse primer including SEQ ID NO 2.

6. Artificial Calibrating Virus (ACV) in accordance with claim 1, characterized by the fact that the genomic region of the HIV used for its development being conservative and the fluorescent probe including SEQ ID NO. 3.

7. Artificial Calibrating Virus (ACV) in accordance with claim 1, characterized by the fact that the synthetic probe includes SEQ ID NO. 6.

8. Artificial Calibrating Virus (ACV) in accordance with claim 1, characterized by the fact that it is not infectious by the deletion of part of its genome, without change in its structure and in its characteristics.

9. Artificial Calibrating Virus (ACV) in accordance with claim 1, characterized by the fact that it is used for the quantification of the viral load by means of the use of the real-time PCR technique.

10. Artificial Calibrating Virus (ACV) in accordance with claim 9, characterized by the fact that the artificial mutation changes the genome or a part thereof or any virus to generate a region that is different from the original genome, but with the same complexity of base pairs.

11. Artificial Calibrating Virus (ACV) in accordance with claims 9 and 10, characterized by the region mutated of the viral genome, target of detection between the natural viral populations and the respective artificial calibrating virus.

12. Kit for the quantification of the viral load of a patient infected by the HIV virus, characterized by the fact that it uses the artificial calibrating virus of SEQ ID NO. 7.

13. A method to quantify the viral load of an individual characterized by the fact that the referred to method includes the:
generation of a calibrating virus through artificial mutation as of a parallel reaction with the natural virus, being this artificial virus non infectious by the deletion of part of its genome, not changing its structure and its physical and chemical characteristics;
alteration by artificial mutation of a virus genome or genomic region of any virus with the purpose of generating a region different from its original natural genome, having the same complexity, as long as it is possible to use this region as preferential and differential target for the detection between the natural viral populations and respective artificial calibrating virus
amplification by PCR-rt of the calibrating virus and the natural virus, and
quantification of the detection differential since there is no base complementation between the natural and artificial sequence generated, through the use of two different and specific oligonucleotides, for each one of the viruses.
